(19) United States
(12) Patent Application Publication
(10) Pub. No.: US 2006/0100675 A1
(43) Pub. Date: May 11, 2006

(54) Device and method for phototherapy of jaundiced infants

(76) Inventor: Steven Gardner, Columbia, SC (US)

Correspondence Address: B. CRAIG KILLOUGH, P. O. DRAWER H, CHARLESTON, SC, 29402, US

(21) Appl. No.: 11/269,922

(22) Filed: Nov. 9, 2005

Related U.S. Application Data
(60) Provisional application No. 60/626,169, filed on Nov. 9, 2004.

(51) Int. Cl. A61N 5/06

(52) U.S. Cl. 607/88

(57) ABSTRACT

A phototherapy device and method for treating neonatal hyperbilirubinemia (jaundice) and related conditions. The device comprises a flexible material encasing flexible circuitry that allows the device to flex as an infant is positioned on it, while also protecting the circuitry. The circuitry comprises a plurality of light emitting diodes (LEDs) mounted within flexible circuitry, means for altering the duty cycle of the LEDs, and wiring to connect the circuitry to a power supply.

Applicant claims the benefit of U.S. Provisional Application Ser. No. 60/626,169 filed Nov. 9, 2004.

### FIELD OF THE INVENTION

This invention relates generally to the treatment of neonatal hyperbilirubinemia jaundice), and more specifically it relates to phototherapy treatment methods and devices.

### BACKGROUND OF THE INVENTION

Approximately 60% of infants born in the United States each year become clinically jaundiced. Jaundice, or hyperbilirubinemia, results from increased production and transiently impaired elimination of bilirubin. While most affected neonates recover rapidly, some infants show persistently high levels of unconjugated bilirubin. Such high levels can lead to kernicterus, a condition involving deposition of bilirubin in the brain, which leads to deficits in cognition, neuromuscular tone and control, and hearing, and even death. The most common therapy for neonatal hyperbilirubinemia is phototherapy. It is estimated that as many as 400,000 neonates in the United States receive phototherapy every year. Phototherapy facilitates the transformation of unconjugated bilirubin to compounds that are more easily excreted.

Phototherapy for treating hyperbilirubinemia is commonly delivered using fluorescent lamps suspended above the neonate. However, fluorescent lamps generate significant heat (infrared radiation), which prevents their placement close to the infant, thereby decreasing the irradiance. Fluorescent light is of a broad spectral range, and cannot be produced in the narrow wavelength range desired. Conventional phototherapy devices typically illuminate the newborn only from above, and do not therefore make optimal use of the available skin area.

The use of fluorescent lamps for phototherapy leads to adverse side effects in many newborns. Such side effects include increased insensible water loss, hypothermia, loose and frequent bowel movements, tanning, and potential nasal obstruction by the eye pads required for preventing retinal damage. Furthermore, there are concerns that phototherapy using fluorescent lamps has potentially harmful effects on biological rhythms, and may increase the incidence of skin cancer in neonates subject to repeated treatment.

### SUMMARY OF THE INVENTION

The present invention provides a phototherapy device and method for treating neonatal hyperbilirubinemia (jaundice) and related conditions, such as Crigler-Najjar Syndrome. The present invention is an improved phototherapy device and method for treating neonatal hyperbilirubinemia. The device comprises a flexible material encasing flexible circuitry which allows an infant to be held and carried by a caregiver while the infant is undergoing treatment. The circuitry comprises a plurality of light emitting diodes (LEDs) mounted in a pattern, flexible circuitry, means to alter the duty cycle of the LEDs, and wiring to connect the circuitry to a power supply.

### DESCRIPTION OF THE DRAWINGS

FIG. 1 is an exploded view of the larger elements of the device, with an infant in position for use.

FIG. 2 is an exploded view demonstrated the device as used with an infant.

FIG. 3 is a top plan view of the LED light panel.

FIG. 4 is a side view of the LED light panel.

FIG. 5 is a side view of an additional embodiment of the LED light panel.

### DETAILED DESCRIPTION OF THE PREFERRED EMBODIMENT

Although the following detailed description contains many specifics for the purposes of illustration, and variations and alterations to the following details are within the scope of the invention. Accordingly, the following embodiments of the invention are set forth without any loss of generality to, and without imposing limitations upon, the claimed invention.

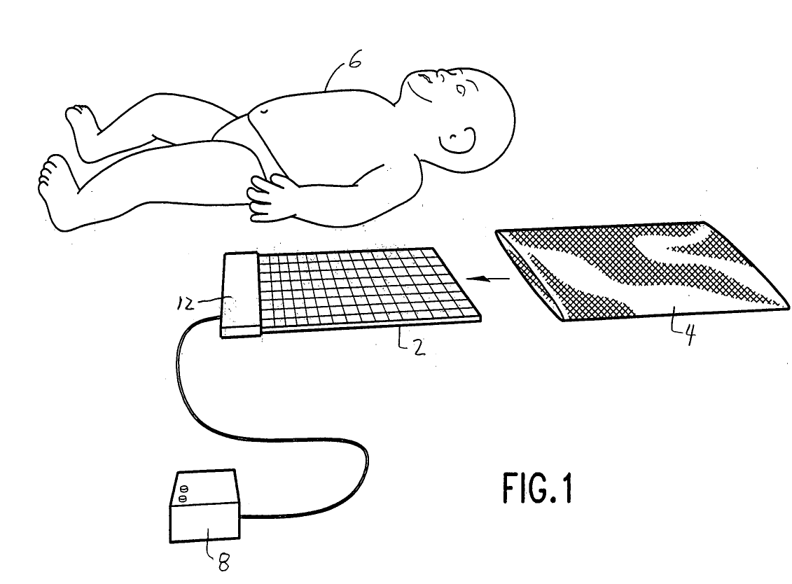

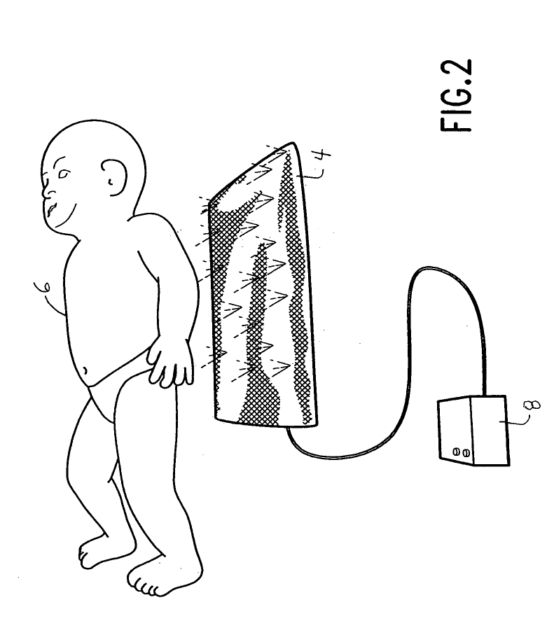

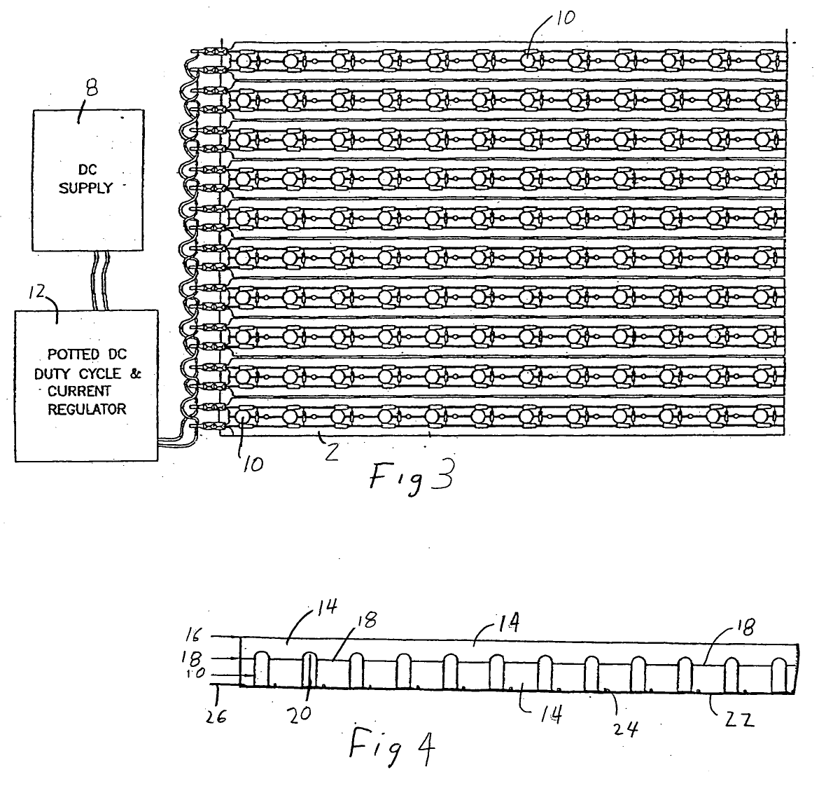

FIG. 1 shows the phototherapy panel 2 with a cover 4. The panel is preferred to be slightly larger than the length of the back of an infant 6. A power supply unit 8 powers the device. As shown in FIG. 2, the sheath allows the light transmitted from the panel to be transmitted through the sheath to the infant's back.

In one embodiment, the phototherapy panel comprises a flexible backing material, a transparent liner, and a flexible circuitry substrate, with light emitting diodes (LEDs) mounted to the flexible circuitry substrate and conductively connected to a power supply. An infant 6 is placed over the panel, with the LEDs emitting light toward the infant's back. The panel is adapted to provide light exposure by means of the LEDs over 100% of the infant's back, or somewhat beyond the infant's back. The phototherapy panel will effectively treat neonatal hyperbilirubinemia, also known as jaundice, via phototherapy.

It is preferred that the portion of the panel 2 in which LEDs are present is wider and longer than the infant's back. However, the panel is preferred to not be substantially longer or wider than is necessary to provide phototherapy to the infant's back. The portion of the panel comprising LED's (the treatment area) are preferred to not be larger than 12 cm by 18 cm. The thickness of the panel is preferred to be 1-2 cm.

The panel as constructed is preferred to be easily portable, that is, the infant may be carried with panel in place against the infant's back. The infant may be comforted and/or transported by a caregiver while the device is in use. Accordingly, the device is constructed so that it is as small as possible, while still providing efficacy. Further, the device as constructed in the preferred embodiment does not need cooling, such as by fans, but it is preferred to keep the number of LEDs to a minimum number that is efficacious so that heat is not detrimental to the device or the infant.

The LEDs are preferred to emit high-intensity blue light suitable for treating neonatal hyperbilirubinemia. The LEDs are arranged to optimize intensity and coverage. The unit is preferred to have not fewer than one LED per 2.5 square centimeter within the matrix where LEDs are present.

A portable power supply 8 is provided. The portable power supply may be one or more batteries that supply direct current to the LEDs, and enable the unit to be highly portable. The power supply is low voltage direct current in the preferred embodiment, thereby reducing the risk of harmful electric shock to the patient. Silicon chips enable the LEDs to have their duty cycle altered from 100% actuation time to as little as 10%, thereby increasing battery life, as well as decreasing the amount of heat generated.

The invention provides doctors and parents with a new device that combines the benefits of a fiber-optic panel and a phototherapy bed most commonly called a “bilibed”. The greatest benefit of the fiber-optic panel is that that the parent is able to hold the infant without interrupting treatment, thereby supporting parent-infant bonding. The benefit of the “bilibed” is that it covers more of the infants back due to its size. The invention is highly portable, has a dense coverage pattern, and intensity levels potentially exceeding 120 μW/cm²/nm (microwatts per centimeter squared per nanometer), which is nearly double the current output of a neonatal fiber optic panel or a phototherapy bed while supporting parent-infant bonding, as well as maintaining a high coverage area.

The use of light-emitting diodes (LEDs) is used in a preferred embodiment to deliver light directly to the neonate's skin. The LEDs are very small, very durable, and long lasting. As a result, the panel comprising the LEDs is portable, lightweight, comfortable, easy to use, and relatively inexpensive. LEDs deliver relatively high light intensity for their physical size and weight, with relatively low power consumption (e.g., 70 mW), and therefore have high efficiency (optical output power/electrical input power). They produce no harmful UV radiation and negligible heat (infrared). The LEDs may emit light of a wavelength suitable for treating hyperbilirubinemia in neonates, such as 420-500 nm, preferably between 440 and 480 nm. Each lens type LED is preferred to emit light at an angle of 90 to 110 degrees.

In a preferred embodiment, the present invention uses lens type LEDs. A phototherapy panel is made of a flexible material that incorporates the LEDs. The panel has a lining inside surface of reflective Mylar that reflects ambient light that is previously reflected from the neonate's skin. A liner seals the panel for hygienic purposes. For example, the liner may be crimped (heat-sealed), stitched, or molded to material.

Because the panel directly contacts the jaundiced neonate, it is highly desirable for the panel to be capable of easy cleaning and sterilization. The material is preferred to be impervious to the solution, solvent, or gas used for cleaning or sterilization. All electronics, including the LEDs, are preferred to be encapsulated within a flexible material, such as a silicone gel 14. The gel may be poured over the LED's and molded to yield a flexible, top planar support surface over which the infant rests. The gel fully encapsulates the tops of the LEDs. The gel is preferred to be transparent or substantially transparent to the selected efficacious wavelength of the LED's. The encapsulating gel simplifies cleaning, and limits the surface area that can harbor bacteria, while protecting the LEDs from impact, liquids (from the infant, or otherwise), and cleaning materials and abrasives. Additionally, the panel liner may be coated with an anti-bacterial coating, such as MEDIGARD, manufactured by the Hydrogiene Corporation of San Diego, Calif. Medigard is formulated to kill a wide spectrum of bacteria, is resistant to cleaning and sterilization processes, and has a reputed four-year killing period. It can be coated in such a way as to be impossible to detach or destroy when applied to materials. Alternatively, the panel cover may be disposable, thereby alleviating the need for cleaning and sterilizing the liner.

As shown in FIG. 4, the tops of the plurality of the LEDs are encapsulated by the substantially transparent gel 14. A reflective sheet 18 surrounds the plurality of LEDs 10. The reflective sheet is below the tops 20 of the LEDs so that light from the LEDs is reflected toward the top planar surface 16 of the panel. The reflective sheet is preferred to be not be materially electrically conductive, and may be MYLAR having a silver colored top surface. The reflective sheet may have a plurality of holes formed therein that are spaced to accept the LEDs within the holes. The flexible circuitry substrate 22 is flexible, and has conductors 24 printed thereon that provide power to the LEDs. The gel also surrounds the LEDs below the reflective sheet and above the flexible circuitry substrate, so that the LEDs are encapsulated, with no spaces or pockets having a material size around or above the LEDs, other than perhaps air bubbles from the formation process.

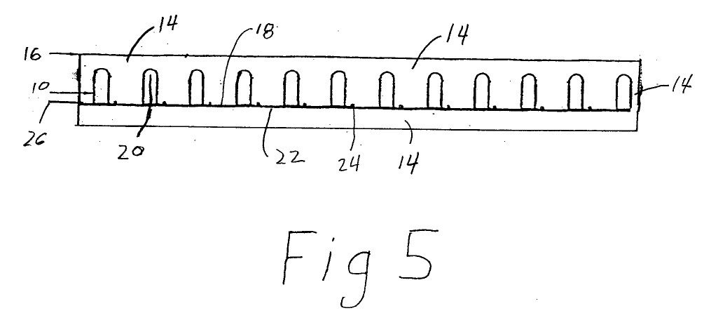

As shown in FIG. 5, which is an additional embodiment from the embodiment of FIG. 4, the plurality of the LEDs 10 are encapsulated by the substantially transparent gel 14. A reflective sheet 18 surrounds the plurality of LEDs. The reflective sheet is adjacent to and above the flexible circuitry substrate, so that light from the LEDs is reflected toward the top planar surface 16 of the panel. The reflective sheet is preferred to be not be materially electrically conductive, and may be MYLAR having a silver colored top surface. The non-conductive reflective sheet may be in contact with the flexible circuitry substrate. The reflective sheet may have a plurality of holes formed therein that are spaced to accept the LEDs within the holes. The flexible circuitry substrate is flexible, and has conductors printed thereon that provide power to the LEDs. The gel is also present below the reflective sheet and the flexible circuitry substrate. All circuitry and the LEDs are encapsulated by the gel, other than the power supply and leads 26 to the power supply. No spaces or pockets of material size are around or above the LEDs, other than perhaps air bubbles from the formation process. Lead 26 extends from the gel material and communicates with the LEDs by a printed circuit on the flexible circuitry substrate.

Each panel is preferred to contain not more than 70 low profile lens type LEDs that do not exceed 2.5 mm in height. The LEDs are mounted on a flexible circuit substrate, and are connected to a circuit bus that communicates with the duty cycle and current regulator 8, and the power supply 10. This device is preferred to have an irradiance level of at least 80 μW/cm²/nm. The lens type LED's provide higher intensity levels than LEDs without lenses.

The power supply connection is preferred to be a waterproof or moisture-proof electrical plug that attaches the electrical cord to the power supply. The power cord may be removed for cleaning by unplugging the cord from the waterproof connector. Even though connection protrudes from the panel, cleaning solutions cannot enter the panel.

The power supply may be a standard portable power supply capable of generating a relatively low DC voltage, such as 9V or 12V provided by a battery, including a rechargeable battery. Low voltage is desirable for reducing the risk of flexible, and has conductors printed thereon that provide power to the LEDs. The gel is also present below the reflective sheet and the flexible circuitry substrate. All circuitry and the LEDs are encapsulated by the gel, other than the power supply and leads 26 to the power supply. No spaces or pockets of material size are around or above the LEDs, other than perhaps air bubbles from the formation process. Lead 2 extends from the gel material and communicates with the LEDs by a printed circuit on the flexible circuitry substrate.

Each panel is preferred to contain not more than 70 low profile lens type LEDs that do not exceed 2.5 mm in height. The LEDs are mounted on a flexible circuit substrate, and are connected to a circuit bus that communicates with the duty cycle and current regulator 8, and the power supply 10. This device is preferred to have an irradiance level of at least 80 μW/cm²/nm. The lens type LED's provide higher intensity levels than LEDs without lenses.

The power supply connection is preferred to be a waterproof or moisture-proof electrical plug that attaches the electrical cord to the power supply. The power cord may be removed for cleaning by unplugging the cord from the waterproof connector. Even though connection protrudes from the panel, cleaning solutions cannot enter the panel.

The power supply may be a standard portable power supply capable of generating a relatively low DC voltage, such as 9V or 12V provided by a battery, including a rechargeable battery. Low voltage is desirable for reducing the risk of electrocution for the neonate under treatment. Appropriate circuitry is provided to control the current to the LEDs.

The current supplied to LEDs is not constant in one embodiment, but is rather pulsed, with a duty cycle of 10-50%. The power supply contains appropriate electronic circuitry to control the timing and activation of LEDs. For example, a circuit 12 providing a direct current duty cycle and current regulator may be provided for intermittent operation. Operating LEDs at such a low duty cycle allows them to be overdriven, i.e., operated with a higher applied current than would be feasible with a constant current. The LEDs are not overdriven because the output of the lens type LED is far brighter than that of the surface mount type used by the current artwork device. This also alleviates the need for a heat sink to pull heat away from the device. The allowable ranges for duty cycle, forward current, and pulse width are determined by the operating characteristics of the particular LEDs that are used, according to information provided by the LED manufacturer. The power supply preferably has external controls that allow a person supervising the phototherapy to control the light intensity, frequency (i.e., pulse width), and duty cycle. It may instead have preprogrammed control circuitry that regulates the exposure time and other variables of the treatment.

The intermittent power may be supplied with any pulse width satisfying the LEDs' operating constraints. Intermittent operation at higher power provides significantly more efficient bilirubin photoconversion than does constant operation at lower power. Bilirubin is produced as a by-product of the break down of hemoglobin, which is at high levels after birth. After birth, excess hemoglobin is broken down in a short period of time thereby causing high bilirubin levels (hpyerbilirubinemia). Bilirubin enters the skin in significant amounts when the bilirubin concentration is quite high, leading to the characteristic yellowish skin tone; phototherapy is believed to affect bilirubin in the skin only. When the light is off, the bilirubin concentration in the skin gradually builds up, and it is then converted when the light is on again. Therefore, consistent phototherapy with high irradiance levels is necessary to reduce the overall bilirubin concentrations in the neonate.

The present invention allows the neonate to be fully covered during phototherapy via a gown or blanket, and therefore, the neonate is much more comfortable and less susceptible to hypothermia. The neonate may be wrapped in a blanket, with the present invention inside the blanket against the child's back. The device may also be placed inside clothing. The flexible plastic casing allows deformation of the device as the child is held within a blanket, without damaging or adversely affecting the performance of the device.

In one embodiment, a power supply 8 includes a battery, which may be replaceable or rechargeable, that supplies power to LEDs. The battery or batteries may be held in a pocket that is attached to the outside of the gel material, with appropriate circuitry for pulsing the LEDs encapsulated in the gel material. The use of a battery makes the panel fully portable, allowing it to be used when the neonate is transported to and from, and within, the hospital, for example.

Treatment time using the phototherapy device of the present invention varies widely depending upon the conditions of the particular neonate, particularly the serum bilirubin level. However, treatment times are less than with prior methods, which provide much lower irradiance and less surface exposure. The LEDs may be arranged in various configurations. LEDs emitting wavelengths of light other than blue wavelengths may be used, and multiple different wavelengths may be used on the same panel. Phototherapy has been shown to cause a decrease in the endogenous levels of riboflavin, a natural photosensitizer. When irradiated by broad wavelength white or blue light, riboflavin produces singlet oxygen that may react with bilirubin and other organic compounds, causing photodamage and decreased riboflavin levels in developing neonates. Narrow wavelength blue to green LED light is less likely to be absorbed by endogenous photosensitizers and appears to produce fewer side effects, and thus LEDs with higher wavelengths (blue to green) may be included in the device of the present invention.

The device is preferred to be flexible in both the longitudinal and transverse direction. In this embodiment, a substantially transparent pourable silicon plastic or gel is poured over the device to encase the circuitry, and is allowed to set. In particular, the LEDs are covered with the casing formed by the plastic or gel. The casing serves as structural support to counter the weight of the patient, thereby preventing the LEDs from being damaged or moved out of proper alignment for maximum efficacy, as well as insuring patient safety and protection from the electrical circuitry. The encasing material is flexible, and allows the device to be deformed so as to follow contours of the infant's body, particularly as the infant is held.

A substantially transparent removable sheath, which is preferred to be non-latex, may contain the device. A cover 4 having a large mesh that will permit transmission of light through the mesh may be used in place of, or in addition to, the sheath. The sheath or cover may be removed and cleaned, or disposed of and replaced, for disinfection purposes.

What is claimed is:

1. A phototherapy device for treating neonatal hyperbilirubinemia, comprising:
flexible circuitry, said flexible circuitry comprising a plurality of light emitting diodes mounted in a pattern,
a flexible material surrounding and encasing each of said light emitting diodes that comprises said plurality of light emitting diodes, and
a power supply for powering said LEDs.

2. A phototherapy device for treating neonatal hyperbilirubinemia as described in claim 1, wherein said flexible circuitry is surrounded by and is encased in said flexible material.

3. A phototherapy device for treating neonatal hyperbilirubinemia as described in claim 1, wherein said each of said light emitting diodes that comprises said plurality of light emitting diodes are surrounded by a sheet of reflective material that is encased in said flexible material, and wherein said reflective material is below a top of each of said light emitting diodes that comprises said plurality of light emitting diodes.

4. A phototherapy device for treating neonatal hyperbilirubinemia as described in claim 1, wherein said each of said light emitting diodes that comprises said plurality of light emitting diodes are surrounded by a sheet of reflective material that is encased in said flexible material, and wherein said reflective material is below a top of each of said light emitting diodes that comprises said plurality of light emitting diodes, and wherein said reflective material is positioned over said flexible circuitry.

5. A phototherapy device for treating neonatal hyperbilirubinemia as described in claim 1, wherein said plurality of light emitting diodes are lens type light emitting diodes.

6. A phototherapy device for treating neonatal hyperbilirubinemia as described in claim 1, wherein said flexible material comprises silicone gel.

7. A phototherapy device for treating neonatal hyperbilirubinemia as described in claim 1, wherein said flexible material is substantially transparent to an efficacious wave length of light emitted by said plurality of light emitting diodes.

8. A phototherapy device for treating neonatal hyperbilirubinemia as described in claim 1, wherein said flexible material is covered by a sheath, and wherein said sheath is substantially transparent to an efficacious wave length of light emitted by said plurality of light emitting diodes.

9. A phototherapy device for treating neonatal hyperbilirubinemia as described in claim 1, wherein said plurality of light emitting diodes is controlled by a circuit that operates said plurality of light emitting diodes on an intermittent basis.

10. A phototherapy device for treating neonatal hyperbilirubinemia as described in claim 9, wherein said flexible circuitry and said circuit that operates said plurality of light emitting diodes on an intermittent basis is surrounded by and is encased in said flexible material.
